(19) United States
(12) Patent Application Publication
(10) Pub. No.: US 2024/0118287 A1
(43) Pub. Date: Apr. 11, 2024

(54) ANALYSIS PLATE FOR A MASS SPECTROMETRY ANALYSIS AND ASSOCIATED METHOD FOR CHARACTERIZING MICROORGANISMS BY MASS SPECTROMETRY

(71) Applicant: BIOMÉRIEUX, Marcy l'Etoile (FR)

(72) Inventor: Jean-Philippe CHARRIER, Tassin la demi Lune (FR)

(73) Assignee: BIOMÉRIEUX, Marcy l'Etoile (FR)

(21) Appl. No.: 18/273,340

(22) PCT Filed: Feb. 4, 2022
(86) PCT No.: PCT/FR2022/000008

§ 371 Date: Jul. 20, 2023

(30) Foreign Application Priority Data
Feb. 4, 2021 (FR) FR2101049

(51) Int. Cl. G01N 33/68; H01J 49/04

(52) U.S. Cl. CPC G01N 33/6851; H01J 49/0418

(57) ABSTRACT

An analysis plate configured to allow a characterization of microorganisms by mass spectrometry, wherein the analysis plate includes at least one analysis zone configured for a biological sample containing a population of at least one microorganism, wherein all or part of the analysis zone is made of a porous material, the analysis zone including at least one antimicrobial agent.

The present invention relates to the field of microbiology. More precisely, the invention relates to the characterization of a population of at least one microorganism using mass spectrometry, and in particular, mass spectrometry using a MALDI ionization technique (MALDI: Matrix-Assisted Laser Desorption/Ionization).

The MALDI technique combined with a mass analyzer of the time of flight (TOF) type, has been in use for some years for performing rapid identification of microorganisms, at least at the species level. Various systems suitable for this type of characterization are marketed by the applicant, as well as by the companies Bruker Daltonics, ASTA and Bioyang.

A microorganism is identified from the MALDI-TOF mass spectrum of the most abundant proteins in the microorganism, by comparing with reference data allowing identification of the genus and most often the species of the microorganism. The protocol used routinely comprises depositing at least a portion of a colony of the microorganism on an analysis plate, adding a matrix that is suitable for the MALDI technique, acquiring the mass spectrum and identifying the species by comparing with reference data stored in a database (Welker M, Moore ER. Syst. Appl. Microbiol. 2011 February; 34(1):2-11, Applications of Whole-Cell Matrix-Assisted Laser-Desorption/Ionization Time-Of-Flight Mass Spectrometry in Systematic Microbiology).

The MALDI-TOF technique has also been used for detecting a microorganism's resistance to an antibiotic. Thus, methods are known for detecting resistance in MALDI-TOF, after contacting with an antibiotic, with or without culture.

The applicant has proposed functionalizing the analysis surface by prior deposition of the antimicrobial agent, said microbial agent being dried after its deposition. The analysis surface, also called target, may thus comprise a set of functionalized zones ready to receive a sample potentially containing microorganisms for characterizing their resistance. This functionalization makes it possible to simplify the procedure in a way that is advantageous for the user. The functionalized targets can be prepared in advance, for example in the factory, to give a consumable that is ready to use. The sample containing at least one microorganism is then simply deposited in liquid form on one of the functionalized zones, incubated, dried and analyzed by MALDI-TOF.

Methods for detecting mechanisms of resistance by MALDI-TOF for any type of antibiotic are also known after culturing the microorganisms in the presence of the antibiotic. These methods measure a change in the bacterial protein pattern, or an increase in bacterial biomass in the course of culture when the microbe is resistant to the antibiotic. More particularly, the increase in biomass can be determined by comparing mass spectra obtained after growth with and without antibiotic, one of the growth conditions having been implemented in a culture medium with heavy isotopes, or else by comparing the signal of the characteristic peaks of the microorganism to be analyzed relative to a reference substance added in a metered form. This last-mentioned method is used in the MBT-ASTRA method described by Sparbier et al. (Sparbier et al., 2016, Methods, 104:48-54, MBT-ASTRA: a suitable tool for fast antibiotic susceptibility testing?). In practice, 200 μl of microorganisms at 0.5 McFarland are cultured at 37° C. with or without antibiotic for a predetermined time. After incubation, the cells are sedimented by centrifugation and washed successively with 150 μl of pure water and then 100 μl of 70% ethanol. They are then lysed with 10 μl of 70% formic acid and then 10 μl of pure acetonitrile containing the reference substance. The solution of lysed cells is deposited on the MALDI-TOF analysis plate for acquiring a mass spectrum and comparing the intensity of the peaks of the proteins of the microorganism against that of the reference substance. An algorithm finally makes it possible to classify the microorganism as resistant or sensitive to the antibiotic depending on a certain threshold.

Idelevich et al. recently simplified this method by carrying out incubation of the solution of microorganisms with or without antibiotic directly on the target. The new method is called DOT-MGA (direct-on-target microdroplet growth assay) (Idelevich et al., Clin Microbiol Infect. 2018 July; 24(7):738-743, Rapid Detection of Antibiotic Resistance by MALDI-TOF Mass Spectrometry Using a Novel Direct-on-Target Microdroplet Growth Assay). However, it has several prohibitive drawbacks, in particular the need to incubate a drop of liquid that must not dry out, and the need to remove the excess liquid by blotting.

The step of incubation of the sample, optionally comprising microorganisms with or without antibiotic, must imperatively be carried out in a liquid medium, controlling the composition of the medium. This incubation may take several hours. Now, a drop of some microliters dries out the more quickly in the open air the higher the temperature and the drier the air. When the drop dries, the composition of the liquid medium changes: the concentration of salts and nutrients increases and may finally inhibit the growth of the microorganism to be characterized. A resistant isolate might thus not grow, and give the illusion that the microorganism is sensitive to the antibiotic tested. It is therefore imperative to carry out incubation in a humid atmosphere, and if possible in a moisture-saturated atmosphere, to limit evaporation to the greatest possible extent.

The Bruker company has tried to overcome this difficulty by proposing a humid chamber, maintaining humidity by deliquescence of a suitable substance (WO 2019/011746 A2). However, maintaining a humid atmosphere presents the drawback of promoting the development of spore-forming molds, which will contaminate the air and all the surfaces of the humid chamber or of the stove. The spores thus formed will contaminate the drops during their incubation and distort the result of the analysis, either by disturbing the growth of the microorganisms to be characterized, or by contributing to the appearance of artifact peaks on the mass spectrum.

The step of blotting the liquid drop present on the target at the end of the incubation step is also a difficult operation. It requires great care to avoid contaminating two adjacent wells, which are generally very close. The blotting paper must not touch the surface, so as not to risk removing, by scratching or wiping, the microorganisms that may be present there. Otherwise the blotting paper may entrain some of the microorganisms, which distorts the quantitative analysis of their growth rate. Moreover, the blotting paper may be contaminated with pathogenic microorganisms. It must therefore be handled and removed as potentially soiled with infectious microorganisms, which is potentially dangerous for operator safety.

Furthermore, the blotting step is currently carried out by hand, and therefore its reproducibility is insufficient from one operator to another for general routine use in clinical laboratories. Moreover, in the case of automation, potentially infectious blotting paper may contaminate the robot, from falling droplets, or if their discharge creates aerosols.

The Bruker company has tried to overcome these difficulties by proposing a method of extraction using an absorbent material comprising a pattern of indentations or holes, in patent application EP 3 376 202 A1. The arrangement of the latter corresponds to the predefined positions of the droplets of samples on the target. In this way the edges of the indentations or holes are assumed to come into contact with the droplets so as to blot them progressively by one of the sides of the droplets. However, this method is still difficult to implement. The droplets are not necessarily positioned perfectly identically from one receiving zone to another. They may be slightly off-center, which makes correct contact difficult between the absorbent material and the droplet. Consequently, blotting may be nonuniform and of low reproducibility.

The aim of the invention is to overcome some or all of the aforementioned drawbacks and in particular facilitate the characterization of microorganisms present in biological samples by limiting transfers from one support to another and by functionalizing the analysis plate.

For this purpose, the invention relates to an analysis plate configured to allow characterization of microorganisms by mass spectrometry, said analysis plate comprising at least one analysis zone configured for a biological sample containing a population of at least one microorganism, characterized in that part or the whole of said analysis zone is made of a porous material, and said analysis zone comprises at least one antimicrobial agent.

The analysis plate thus configured makes it unnecessary to use different supports for incubation and ionization. Moreover, owing to its functionalization by the prior deposition of a microbial agent, it is possible to characterize a microorganism and ascertain its resistance. Finally, the at least one porous analysis zone allows filtration of the excess liquid while retaining the microorganisms within the analysis zone and maintaining humidity that is favorable for incubation. In fact, some microliters of liquid optionally containing microorganisms and at least one antimicrobial agent can be incubated for several hours on the analysis plate according to the invention without it drying out.

Moreover, the use of an analysis plate comprising a set of analysis zones made of porous material makes it possible to perform the steps of incubation and of filtration together for all of the samples analyzed on the analysis zones, which is particularly advantageous from an ergonomic standpoint for ensuring a high analysis throughput.

According to a characteristic feature of the invention, the analysis plate according to the invention comprises several analysis zones.

Advantageously, each analysis zone forms a “spot” or a target, preferably of circular shape.

According to a characteristic of the invention, the surface of the analysis plate is conductive, in order to promote subsequent ionization, at least at the level of the analysis zone or zones.

For example, the analysis plate according to the invention is formed from a polymer such as polypropylene, said polymer being covered with a layer of stainless steel. Furthermore, the polymer may contain a conductive material such as carbon black.

According to a characteristic feature of the invention, the analysis plate according to the invention comprises between 48 and 96 analysis zones.

According to a characteristic feature of the invention, the analysis plate according to the invention further comprises at least one, or even two or three reference analysis zones, which may be of different size relative to analysis zones. These reference analysis zones serve as zones for calibration and/or zones for validation of the analyses.

In the context of the invention, “analysis zone” denotes a zone intended to receive a biological sample to be analyzed, said zone being made of a porous material and comprising at least one antimicrobial agent.

“Porous material” means a matrix containing small pores or cavities, which may contain one or more fluids (liquid or gas).

According to a characteristic feature of the invention, the porous material may be “open”, i.e. the pores are connected together, forming very fine channels.

According to a characteristic feature of the invention, the porous material of the analysis zone or zones is a material with capillary porosity, hygroscopic, and permeable to the air, which makes it possible to filter, at least partially, the substances deposited on the analysis zone. Advantageously, the porous material according to the invention thus makes possible the absorption of water or of water vapor and the passage of air.

According to a characteristic feature of the invention, the porous material of the at least one analysis zone is a filtering material.

According to a characteristic feature of the invention, the pore size of the porous material of the analysis zone is less than the size of said at least one microorganism to be characterized. Thus, there is no risk of said at least one microorganism passing through the pores of the analysis zone, and it therefore remains in the analysis zone in order to be characterized. In fact, owing to the choice of the porosity of each analysis zone, the microorganisms are retained naturally on the surface, without being able to pass through it, the bottom of the analysis plate thus remaining devoid of any potentially infectious microorganism.

In the context of the present invention, “pore size” means the diameter of the pore. All the pores in one and the same analysis zone may be of the same size or of different sizes. Thus, in the context of the invention, the pore with the largest size in an analysis zone is smaller than the size of the microorganism to be characterized.

According to the invention, when an antimicrobial agent is deposited on said analysis zone of the analysis plate, the analysis zone is said to be “functionalized”.

“Antimicrobial agent” means a compound capable of decreasing the viability of a microorganism and/or of decreasing its growth or reproduction. Said antimicrobial agents may be antibiotics, when they are directed against bacteria. However, the invention is applicable to any type of microorganism of the type bacteria, yeasts, molds or parasites and therefore to the corresponding antimicrobial agents.

In the context of the invention, the antimicrobial agent may be selected so as to allow identification of resistance to any antimicrobial agent, for example an antibiotic or an antifungal.

According to a characteristic feature of the invention, when the analysis plate comprises several analysis zones, at least two zones, or even more, will bear a different antimicrobial agent. It is thus possible to characterize several populations of microorganism(s) with one and the same analysis plate in a single analysis.

Advantageously, according to the invention, each of the analysis zones may bear a different antimicrobial agent.

According to a characteristic feature of the invention, one and the same antimicrobial agent may be present on at least two separate analysis zones, in order to perform the characterizations in duplicate.

Advantageously, the analysis plates according to the invention will be able to be supplied directly to the user, who will therefore only have to deposit the population of microorganism(s) to be analyzed thereon, then after an incubation step, deposit a matrix thereon. Said analysis plates will be able to be marketed in individual packaging or packaging comprising several plates.

The present invention also relates to a system comprising:
an analysis plate according to the invention, on which a population of at least one microorganism and a culture medium are deposited on at least one analysis zone and
an incubation element configured for maintaining the humidity of the at least one analysis zone,
said incubation element being positioned under the analysis plate.

Owing to this configuration, the incubation element makes it possible to avoid drying out of the drop or drops of liquid culture medium, of the microorganism population and of the antimicrobial agent or of their being aspirated by capillarity by the porous material of the at least one analysis zone, and we also avoid having recourse to a humid chamber, which would make the method more complicated.

According to a characteristic feature of the invention, the incubation element is a moisture-saturated gas or a liquid, for example ultrapure water or physiological saline solution, i.e. water containing salts for ensuring osmotic pressure and pH that are suitable for the physiology of the microorganism to be characterized.

According to a characteristic feature of the invention, the system comprises a support formed of a porous material, said support being positioned under the analysis plate.

According to a characteristic feature of the invention, the incubation element is contained in the support and is configured for humidifying said support. Alternatively or additionally, the support is moistened by the culture medium, such as Muller-Hinton culture medium or any other culture medium familiar to a person skilled in the art. This moistening by a culture medium will make it possible to promote microbial growth during incubation of the analysis plate according to the invention.

According to the invention, the porous material of the incubation support may have a porosity equivalent to or different than that of the porous material of the at least one analysis zone.

In the context of the present invention, the support made of a porous material is capable of absorbing a certain quantity of liquid by capillarity. As examples of support made of a porous material, we may mention a natural or synthetic sponge, blotting paper, synthetic foam, glass fiber, cotton, sand, sawdust etc. Thus, among the synthetic materials we may mention, as examples, polyethylenes, polyesters, such as polyethylene terephthalate (PET), or polyamides. They may also be copolymers, such as polyethylene/polyester polymers, such as a polyethylene/PET copolymer. When it is a fibrous material, the fibers may consist of a single-component material or a two-component material. A two-component fiber may for example consist of a PET core and a polyethylene skin. Natural porous materials may also be used, for example such as cotton fibers, paper fibers.

According to one embodiment of the present invention, the incubation support is blotting paper.

In the present invention, “blotting paper” means a fibrous material consisting partly or entirely of fibers. These fibers may be assembled in an orderly manner or randomly (i.e. a woven or a nonwoven fabric). This material, which has the capacity to absorb aqueous liquids, may consist of a single type of fiber, or a mixture of fibers, whether or not belonging to the same class. The fibers may be classified according to their chemical composition (mineral or organic) and their origin (natural or artificial). As examples, we may mention glass fiber as artificial mineral fiber or cellulose fiber (derived from cotton or from wood) as natural organic fibers of vegetable origin.

According to a characteristic feature of the invention, the system comprises an incubation chamber. In the present invention, “incubation chamber” means a closed enclosure in which the incubation step can take place without the deposits of sample or microorganism population drying in a way that is deleterious for the analysis. The closed enclosure is configured to maintain a sufficient humidity level for the at least one microorganism population to develop, i.e. to divide and grow, without dehydrating.

Advantageously, the closed enclosure is thermostatically controlled.

According to a characteristic feature of the invention, the closed enclosure may also be arranged inside another enclosure, itself thermostatically controlled.

According to a characteristic feature of the invention, the closed enclosure may be in the form of a cassette, in which the analysis plate will be inserted.

According to a characteristic feature of the invention, the system comprises an aspirating device configured to allow removal of the excess liquid from the surface of the analysis zone.

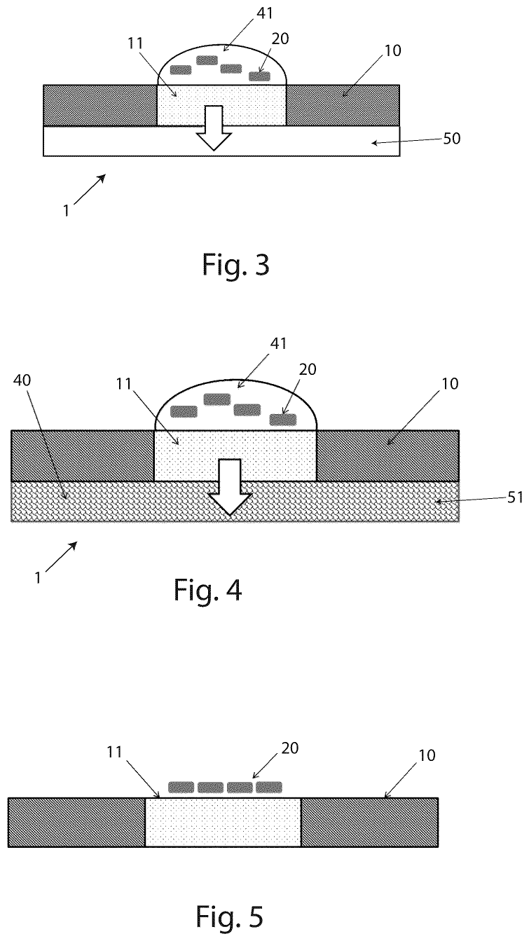

According to a characteristic feature of the invention, the aspirating device 50, 51 may be either in the form of a vacuum aspirating device 50 (FIG. 3) or in the form of a porous support such as blotting paper for example.

According to a characteristic feature of the invention, when the aspirating device is in the form of a support made of porous material it may be identical to the incubation support.

The invention also relates to a method for characterizing a population of at least one microorganism, said characterization comprising at least determination of the possible presence of resistance of said microorganism to at least one antimicrobial agent disposed on the analysis plate according to the invention.

According to the invention, the method for characterizing a population of at least one microorganism, said characterization comprising at least determination of the possible resistance of a population of a microorganism to at least one antimicrobial agent, is characterized in that it comprises the following successive steps:
a step consisting of supplying an analysis plate or a system according to the invention, for characterization of said population of at least one microorganism,
a step of depositing the population of said at least one microorganism in liquid form on said at least one analysis zone in contact with the antimicrobial agent previously deposited on said analysis zone,
an incubation step, consisting of storing said analysis plate in conditions and for a sufficient period to allow interaction of said at least one antimicrobial agent and of said at least one microorganism present,
a step of removing the liquid containing the population of said at least one microorganism by aspiration through the pores of the analysis zone,
a step of depositing, on said at least one analysis zone, a matrix suitable for the MALDI ionization technique, for example of the HCCA type, known to kill the microorganisms present on the surface of the target,
a step of analysis, by mass spectrometry using a MALDI ionization technique, of a population of said at least one microorganism deposited on said analysis zone, making it possible to conclude whether a population of a microorganism resistant to the antimicrobial agent is present in the analysis zone.

In the context of this method, the population of microorganism(s) is deposited on an analysis zone of an analysis plate according to the present invention, and then its characterization is undertaken.

According to a characteristic feature of the invention, deposition is carried out in such a way that the population of microorganism(s) is deposited uniformly in the analysis zone. For this purpose, it will be possible to use the procedures described for carrying out the standard identification of microorganisms, in the manuals for using systems or instruments employing MALDI-TOF technology. Verification that a population of at least one microorganism has indeed been deposited may be done beforehand by a suitable assay, in particular on agar. Preferably, a single population of a single microorganism will be deposited on the analysis zone.

Advantageously, the population of microorganism(s) will be able to come from various sources. As an example of a source of microorganism(s), we may mention specimens of biological origin, in particular animal or human. Said specimen may correspond to a sample of biological fluid, such as whole blood, serum, plasma, urine, cerebrospinal fluid, organic secretion, to a tissue sample or to isolated cells. This sample may be deposited as it is, or will preferably undergo, before being deposited on the analysis zone in question, a preparation of the enrichment or culture type, concentration and/or a step of extraction or purification by methods known by a person skilled in the art. However, said preparation cannot correspond to a step of lysis that leads to disintegration of the microorganisms and loss of their contents before deposition in the analysis zone. The population of microorganism(s) may be deposited in the form of an inoculum. In the context of the invention, the population of microorganism(s) deposited on the analysis zone will preferably be a population of living microorganism(s), although it is not excluded to perform extraction of the population of microorganism(s) from a biological sample, using a detergent, which could affect the viability. In such a case, it may be wise to prolong the incubation step. Furthermore, the source of the population of microorganism(s) may also be a food industry product such as meat, milk, yoghurt and any other consumable product that could be contaminated, or else a cosmetic or a pharmaceutical. Once again, said product may undergo a preparation of the enrichment or culture type, concentration and/or a step of extraction or purification, in order to obtain the population of microorganism(s) to be deposited.

Advantageously, the source of microorganism(s) could have been cultured beforehand in a broth or on agar so as to enrich it in microorganisms. Said media, agar or broth, are familiar to a person skilled in the art. Enrichment on agar is particularly favorable since it makes it possible to obtain colonies of microorganisms that can be deposited directly in the analysis zone. In the context of the invention, it will be possible to deposit preferably a cellular medium comprising a bacterial population in the analysis zone. Preferably, the population of microorganisms deposited contains at least 10⁵ CFU of microorganisms. As an example, it will be possible to deposit from 10⁵ to 10⁹ CFU of a microorganism. It is possible for example to proceed directly to the deposition of a biomass, a drop of a suspension of microorganisms, in ultrapure water, or a buffer, or a culture medium. It will be possible to deposit a colony or a fraction of a colony of a microorganism.

The population deposited will preferably comprise a single species of microorganism. However, deposition of a population comprising various microorganisms in the analysis zone is not excluded. In this case, it will be preferable for the microorganisms to be known to be likely to develop different mechanisms of resistance, so as to know which would display the resistance that would be identified.

In the context of the invention, it is not useful to proceed to a particular preparation of a population of microorganism(s) that would be deposited. In particular, the population of microorganism(s) is deposited without having previously been brought into contact with an antimicrobial agent. In fact, in the context of the invention, it is not necessary to carry out a long, tedious preparation of a sample to be deposited, and the population of microorganism(s) deposited can be prepared without a centrifugation step.

Preferably, the antimicrobial agent is deposited from an aqueous solution of the antimicrobial agent. We thus obtain a so-called functionalized analysis zone bearing an antimicrobial agent.

According to the invention, a buffer suitable for the solubility of the antimicrobial agent, as well as for optimal activity of the mechanism at the origin of the targeted resistance, may also be used for preparing the solution of the anti microbial agent.

According to the invention, a drop, for example of about 1 to 2 microliters, of the antimicrobial solution may be deposited, in such a way that the whole analysis zone is covered.

According to a characteristic feature of the invention, deposition of the microbial agent is followed by a drying operation. For example, the water contained in the solution is then evaporated, for example by simple drying in ambient air at room temperature. The analysis plate may be left at a temperature for example in the range from 17 to 40° C., and in particular at room temperature (22° C.). It is also possible to transfer it to a thermostatically controlled enclosure, for example at 37° C., to speed up drying.

According to a characteristic feature of the invention, it is also possible for the antimicrobial agent to be immobilized in the analysis zone by covalent bonds or bonds with strong affinity.

According to a characteristic feature of the invention, the antimicrobial agent is bound to the analysis zone by electrostatic, ionic, or covalent bonds, with or without using a linker or a bonding partner that is specific (antibodies, recombinant phage proteins) or not, by using biotin/streptavidin interaction previously grafted to the surface of the analysis zone and to the antibiotic, or by any type of bond suitable for the nature of the antimicrobial agent and the surface of the analysis zone.

According to the invention, the manner of bonding or of deposition of the antimicrobial agent will, however, be selected so as not to impede any interaction of the antimicrobial agent with a microorganism. In particular, it will be preferable to immobilize the antimicrobial agent using an adhesive agent, rather than by covalent or affinity bonding, to avoid changes in conformation of the antimicrobial agent and to ensure good accessibility of the latter to the active site of the enzyme that would be generated by the microorganism. In the case when an adhesive agent is used, i.e. an agent that will adhere to the analysis plate and therefore improve immobilization of the antimicrobial agent thereon, a mixture of the adhesive agent and of the antimicrobial agent in aqueous solution will then be deposited. The adhesive agent will be, in particular, a water-soluble polymer. As an example of adhesive agent usable for immobilization of the antimicrobial agent in the analysis zone or zones, we may mention heptakis(2,6-di-O-methyl)-β-cyclodextrin. The adhesive agent will be selected as a function of the antimicrobial agent to be immobilized in the analysis zone. In particular, it will be selected as a function of its mass, so that its presence does not distort subsequent detection by MALDI-TOF mass spectrometry with the aim of determining the presence or absence of the microorganism. A person skilled in the art will adjust the amount of adhesive agent used in order to ensure accessibility of the antimicrobial agent by the population of microorganism(s) when the latter has been deposited. For example, in the case of heptakis(2,6-di-O-methyl)-β-cyclodextrin, a heptakis(2,6-di-O-methyl)-β-cyclodextrin/antimicrobial agent weight ratio from 1/20 to 1/2, preferably from 1/10 to 1/5 may be selected.

According to the invention, the amount of antimicrobial agent deposited on an analysis zone will be adapted by a person skilled in the art, as a function of the antimicrobial agent in question. In fact, the antimicrobial agent is tested at a concentration, or with a concentration range, suitable for its therapeutic usage. This concentration, or this concentration range, is specific to each antimicrobial agent and is generally selected according to the recommendations of the European Committee on Antimicrobial Susceptibility Testing (EUCAST, cf. https://www.eucast.org/clinical_breakpoints/) or of the Clinical & Laboratory Standards Institute (CLSI, cf. https://clsi.org/meetings/microbiology/clsi-and-ast/).

According to a characteristic feature of the invention, the method comprises an additional step of depositing a compound different than the antimicrobial agent.

According to a characteristic feature of the invention, the compound may be configured to accelerate the growth of the microorganisms. This compound may be for example a nutrient medium containing peptones. This compound may be deposited beforehand in the analysis zone in combination with the antimicrobial agent or at any other time point in the preparation of the analysis zone.

For example, in the case of beta-lactam antibiotics, an inhibitor of beta-lactamases may be deposited, in order to characterize an ESBL (extended-spectrum beta-lactamase) phenomenon. In particular, it will be possible to deposit a combination of a beta-lactam antibiotic with a beta-lactamase inhibitor such as clavulanic acid, sulbactam or tazobactam.

After deposition of the population of microorganism(s), the analysis zone bearing both the antimicrobial agent and the population of a microorganism to be characterized, is submitted to an incubation step to allow interaction between the microorganism and the antimicrobial agent and therefore, in the case of the presence of a population of microorganisms resistant to the antimicrobial agent, to allow the phenomenon at the origin of the resistance to develop. In particular, the phenomenon of resistance will allow growth of the microorganism, i.e. its division and its multiplication. This will result in an increase in biomass of the microorganism, which after a suitable incubation time will allow it to be detected by mass spectrometry analysis with an ionization source, MALDI-TOF.

In the context of the invention, the phenomenon responsible for resistance to an antimicrobial agent therefore takes place directly on the analysis plate. The phenomenon responsible for resistance to an antimicrobial agent occurs in a minimal volume corresponding to the characterization zone.

The incubation conditions and time will be adapted by a person skilled in the art as a function of the resistance phenomenon to be characterized. The analysis plate may be left at a temperature for example in the range from 17 to 40° C., and in particular at room temperature (22° C.). It is also possible to incubate the analysis plate, for example at 37° C., to promote growth and the phenomena at the origin of resistance.

In the conditions selected, the incubation time will have to be sufficient to allow subsequent mass spectrometry detection of the resistance phenomenon to be detected. Most often, incubation is carried out for at least 2 hours, more preferably for at least 4 hours, and even more preferably for a time from 4 to 12 hours.

It was found, in the context of the invention, that carrying out said incubation step did not in any way hamper identification of the microorganism. Characterization of this kind is even particularly advantageous when it is combined with detection of the resistance phenomenon. In fact, in the context of a population made up of several species of microorganisms, it is possible that a part of the population, for example species A, is resistant to antibiotic X, whereas another part of the population, for example species B, is resistant to antibiotic Y. By carrying out the method according to the invention it will be possible to identify said species A on the analysis zones containing antibiotic X and said species B on those containing antibiotic Y. It will thus be possible to conclude that the sample contains a heterogeneous population of microorganisms made up of subpopulations belonging to several species, of which at least one species is resistant to antibiotic X and at least one other species is resistant to antibiotic Y. Depending on the clinical context, the microbiologist will then be able to decide whether it is a sample of polymicrobial origin or a sample that has been contaminated, for example during sampling. In the latter case the results are unreliable and sampling must be repeated.

According to a characteristic feature of the invention, the incubation step is carried out in an incubation chamber.

According to a characteristic feature of the invention, a step of removing the excess liquid containing the population of said at least one microorganism is carried out after the incubation step.

Advantageously, the step of removing the excess liquid is carried out by aspiration through the pores of the analysis zone, an aspirating device being positioned under the analysis plate.

According to a characteristic feature of the invention, the method comprises a step of drying the analysis zone. Advantageously, removal of excess liquid contributes to drying.

According to a characteristic feature of the invention, the method further comprises a step of depositing a suitable matrix for the MALDI ionization technique necessary for the analysis. This deposition step is carried out on said at least one analysis zone.

Generally, the matrices used in the MALDI technique are photosensitive and crystallize in the presence of the population of microorganism(s), while preserving the integrity of the molecules and microorganisms present. Said matrices, suitable in particular for the MALDI mass spectrometry technique, are well known and, for example, consist of a compound selected from: 3,5-dimethoxy-4-hydroxycinnamic acid; a-cyano-4-hydroxycinnamic acid, ferulic acid and 2,5-dihydroxybenzoic acid. A great many other compounds are known by a person skilled in the art. There are also liquid matrices that do not crystallize at atmospheric pressure or even under vacuum. Any other compound that will permit ionization of the molecules present in the analysis zone under the effect of a laser beam may be used.

For constitution of the matrix, said compound is dissolved, most often in water, preferably of “ultrapure” grade, or in a mixture of water and organic solvent(s). As an example of organic solvents used conventionally, we may mention acetone, acetonitrile, methanol or ethanol. Formic acid, acetic acid, trifluoroacetic acid (TFA) may sometimes be added. An example of a matrix consists, for example, of 20 mg/mL of sinapic acid in an acetonitrile/water/TFA mixture of 50/50/0.1 (v/v). The organic solvent allows the hydrophobic molecules present to dissolve in the solution, whereas water allows dissolution of the hydrophilic molecules. The presence of acid, such as TFA, promotes ionization of the molecules by proton (H⁺) capture.

The solution that is a constituent of the matrix is deposited directly in the analysis zone and then covers the population of microorganism(s) and the antimicrobial agent that are present on the latter.

Optionally, the method according to the invention further comprises, before the step of ionization of the characterization zone, a step of crystallization of the matrix that is present. Most often, crystallization of the matrix is obtained by leaving the matrix to dry in ambient air, the solvent present in the matrix thus being evaporated, for example by leaving the analysis plate at a temperature for example in the range from 17 to 30° C., and in particular at room temperature (22° C.) for some minutes, for example from 5 minutes to 2 hours. This solvent evaporation allows crystallization of the matrix in which the population of microorganism(s) and the antimicrobial agent are distributed. The analysis plate thus prepared is thus dry enough to avoid splashing or spraying of droplets when it is placed under vacuum in the MALDI ionization source.

The population of microorganism(s) and the antimicrobial agent, placed within the MALDI matrix, forming the characterization zone, are submitted to mild ionization. The laser beam used for ionization will be able to have any type of wavelength favorable to sublimation or evaporation of the matrix. Preferably, an ultraviolet or even infrared wavelength will be used. This ionization will for example be able to be carried out with a nitrogen laser emitting a UV beam at 337.1 nm.

During ionization, the population of microorganism(s) and the antimicrobial agent are submitted to laser excitation. The matrix then absorbs the photonic energy and the restitution of this energy leads to sublimation of the matrix, desorption of the molecules present in the population of microorganism(s) and the antimicrobial agent and the appearance of matter in a state described as plasma. Within this plasma, charge exchanges occur between matrix molecules, microorganisms and antimicrobial agent. For example, protons may be detached from the matrix and transferred to the proteins, peptides and organic compounds present at the level of the characterization zone. This step allows mild ionization of the molecules present, without causing their destruction. The population of microorganism(s) and the antimicrobial agent thus release ions of various sizes. The latter are then analyzed in a mass analyzer. This analyzer may be, for example, an analyzer of the “time of flight” (TOF) type. In a TOF, the ions are accelerated by an electric field and travel freely in a tube at reduced pressure, called a flight tube. The pressure applied during ionization and during acceleration of the ions generated is generally in a range from 10⁻⁶ to 10⁻⁹ millibars [mbar]. The smallest ions will then “travel” more quickly than the larger ions, thus allowing them to be separated. A detector is positioned at the terminal end of the flight tube. The flight time of the ions is used for calculating their mass. A mass spectrum is thus obtained, representing the intensity of the signal corresponding to the number of ionized molecules with one and the same mass-to-charge ratio [m/z], as a function of the m/z ratio of the molecules hitting the detector. The mass-to-charge ratio [m/z] is expressed in thomson units [Th]. Once the target has been inserted in the mass spectrometer, the spectrum of a characterization zone is obtained very quickly, generally in less than a minute.

A method of MALDI-TOF mass spectrometry according to the invention may in particular comprise the following successive steps for obtaining the mass spectrum:
providing a characterization zone comprising the population of microorganism(s) to be studied and at least one antimicrobial agent in a matrix suitable for MALDI spectrometry,
optionally obtaining crystallization of the matrix in which the population of microorganism(s) and the antimicrobial agent are disposed,
ionizing the mixture of population of microorganism(s)/antimicrobial agent/matrix, using a laser beam,
accelerating the ionized molecules obtained owing to a potential difference,
allowing the ionized and accelerated molecules to travel freely in a tube at reduced pressure,
detecting at least a portion of the ionized molecules at tube outlet, in order to measure the time that they have taken to travel through the tube at reduced pressure and obtain a signal corresponding to the number of ionized molecules reaching the detector at a given time point,
calculating the mass-to-charge ratio [m/z] of the molecules detected, so as to obtain a signal corresponding to the number of ionized molecules with one and the same mass-to-charge ratio [m/z], as a function of the m/z ratio of the molecules detected.

In general, the m/z ratio is calculated taking into account prior calibration of the mass spectrometer used, in the form of an equation relating the mass-to-charge ratio [m/z] to the flight time of the ionized molecules in the tube at reduced pressure.

The calibration consists of using a set of molecules or a microorganism that will supply ionized molecules covering the range of mass corresponding to the proposed characterization. The m/z ratios of these ionized molecules will serve as standards, to enable the instrument to measure the masses in an adequate manner.

For identification of the microorganism, the calibration may be carried out starting from a strain of bacteria possessing ionized molecules having m/z ratios covering the range of masses used for identification (typically in the range from 2000 to 20000 Da in the case of yeasts, molds, bacteria or parasites).

Any type of MALDI-TOF mass spectrometer may be used for generating the mass spectrum. These spectrometers comprise:
i) an ionization source (generally a UV laser) intended for ionizing the mixture of population of microorganism(s)/antimicrobial agent/matrix;
ii) an accelerator of the ionized molecules by application of a potential difference;
iii) a tube at reduced pressure in which the ionized and accelerated molecules travel;
iv) a mass analyzer intended to separate the molecular ions formed, as a function of their mass-to-charge ratio (m/z);
v) a detector intended to measure the signal produced directly by the molecular ions.

In the context of the invention, MALDI-TOF analysis is preferably an analysis by linear MALDI-TOF, although an analysis by MALDI-TOF-TOF, MALDI-TOF with reflectron, or any type of mass analyzer linked to a MALDI source is not excluded. As an example, the invention is compatible with the use of instruments with a MALDI source combined with an ion trap (MALDI-IT) or multiple analyzers of the quadrupole (Q) type, ion traps (IT), Orbitraps (O), ion mobility tube (IM) etc. Such instruments may thus have hybrid configurations of the type MALDI-Q-IT-TOF, MALDI-QQQ, MALDI-Q-TOF, MALDI-IT-Q-TOF, MALDI-Q-IT-O etc. Analysis by MALDI TOF-TOF, although more complex, could for example be envisaged in certain cases, in particular to fragment the ions produced and supply information on the nature of said ionized molecules. Equipment suitable for said analysis will be employed.

The step of analysis by mass spectrometry makes it possible to conclude whether the population of a microorganism resistant to the antimicrobial agent is present in the analysis zone.

“Resistance” means a phenomenon according to which a microorganism does not display a decrease in its viability, or a stagnation or a decrease in its growth or its reproduction, when it is exposed to a concentration of an antimicrobial agent that is recognized as being clinically effective with respect to said microorganism in the absence of resistance.

The identification of resistance, from the mass spectrum obtained for a given characterization zone, may be done by the detection, on the mass spectrum obtained, of a given mass peak, or of a change in mass peak relative to a reference mass spectrum, in particular from the mass spectrum of the sample incubated on the reference zone not comprising antimicrobial.

It may also be done by detecting the growth of the microorganism in the presence of the antimicrobial agent. This growth may be characterized by an increase in bacterial biomass during culture. More particularly, the increase in biomass can be determined by comparing the signal of the characteristic peaks of the microorganism to be analyzed relative to a reference substance added in metered form as in the method of Idelevich et al. cited above. Preferably, the quantity of microorganisms deposited before the incubation step may be adjusted so as to generate a signal that is insufficient for identifying the microorganism with confidence. The duration of the incubation step may then be optimized so that the growth of the sensitive microorganisms in the absence of antimicrobial or of resistant microorganisms in the presence or in the absence of antimicrobial generates a signal that is sufficient for identifying said microorganism with confidence. A method of this kind was used by T. Horseman et al., who showed, using an instrument marketed by the applicant, that it is possible to characterize the microorganisms identified, as not sensitive to an antibiotic with a probability greater than or equal to 90%, and the microorganisms that were not identified, as sensitive (Horseman et al. Diagn. Microbiol. Infect. Dis., 2020, 97(4):115093, Rapid Qualitative Antibiotic Resistance Characterization using VITEK MS).

Thus, advantageously, to obtain a robust classification by the method of the invention, several deposits may be made per condition. In particular, when 4 deposits are used per condition, an identification probability greater than or equal to 90%, for at least 3 deposits out of 4, makes it possible to characterize the isolate as resistant, and otherwise as sensitive.

In a particular embodiment of the present invention, the method comprises a step of determination of the resistance of said microorganism to said antimicrobial agent by observing the presence of proteins of the microorganism in quantity such that it is possible to conclude that there is growth of the microorganism despite the presence of said antimicrobial agent during the incubation step.

Alternatively, the method according to the invention will be able to be used in particular for determining the minimum inhibitory concentration for the population of at least one microorganism to be characterized using an increasing range of antimicrobial deposited on different sample zones of the analysis plate.

In the context of the present invention, the minimum inhibitory concentration, or MIC, is the lowest concentration of antimicrobial agent capable of inhibiting in vitro all visible growth of the microorganism population studied at a given temperature and for a defined period of time. This value characterizes the bacteriostatic effect of an antibiotic on a bacterium and more generally of an antimicrobial on a microorganism. The MIC is specific to an antimicrobial agent/microorganism pair, each strain having its own value, as a function of natural resistance and/or acquired resistance for the molecule tested.

In a particular embodiment of the invention, the method of characterization allows identification of the genus, or preferably of the species of a population of a microorganism deposited on the analysis zone.

The microorganisms that can be identified by the method of the invention are any type of microorganisms, pathogenic or nonpathogenic, encountered both in industry and in the clinic, which may be subject to phenomena of resistance to antimicrobial agents. They may preferably be bacteria, molds, yeasts or parasites. The invention is particularly suitable for studying bacteria. As examples of said microorganisms, we may mention Gram-positive bacteria, Gram-negative bacteria and Mycobacteria. As examples of Gram-negative bacteria, we may mention those of the genera: Pseudomonas, Escherichia, Salmonella, Shigella, Enterobacter, Klebsiella, Serratia, Proteus, Acinetobacter, Citrobacter, Aeromonas, Stenotrophomonas, Morganella and Providencia, and in particular Escherichia coli, Enterobacter cloacae, Enterobacter aerogenes, Citrobacter sp., Klebsiella pneumoniae, Klebsiella oxytoca, Pseudomonas aeruginosa, Providencia rettgeri, Pseudomonas putida, Stenotrophomonas maltophilia, Acinetobacter baumannii, Comamonas sp., Aeromonas sp., Morganella morganii, Proteus mirabilis, Salmonella senftenberg, Serratia marcescens, Salmonella typhimurium etc. As an example of Gram-positive bacteria, we may mention those of the genera: Enterococcus, Streptococcus, Staphylococcus, Bacillus, Listeria and Clostridium.

Reference spectra obtained by MALDI-TOF for said microorganisms, corresponding to their predominant proteins, are available and recorded in databases available with commercial MALDI-TOF equipment, and allow the presence of said microorganisms to be identified by comparison.

Identification of a population of a microorganism present will therefore be able to comprise the following steps:
a) providing a reference mass spectrum, for at least one microorganism and most often for a series of microorganisms,
b) submitting the population of microorganism(s) and the antimicrobial agent deposited on the analysis zone and placed in the presence of the matrix (corresponding to a characterization zone according to the invention), to ionization,
c) acquiring a mass spectrum obtained following this ionization, in the range of mass of interest for identification of the microorganism,
d) comparing the mass spectrum obtained in step c) with the reference spectrum and deducing therefrom the genus, or preferably the species, of at least one microorganism.

In the case of identification of microorganisms, a calibration is performed in a range of mass corresponding to high masses, typically in the range from 2000 to 20000 Da, and preferably in the range from 3000 to 17000 Da. The mass spectrum obtained in step c) is also within this range of mass.

The calibration may be carried out by placing a population of a reference microorganism in a reference analysis zone present on the analysis plate and analyzing it by MALDI-TOF mass spectrometry. Said reference microorganism may be, for example, an E. coli bacterium. For this calibration, it will be possible to use the procedures described for carrying out the standard identification of microorganisms, in the operator manuals of the commercial MALDI-TOF instruments, such as the VITEK®MS equipment marketed by the company bioMérieux.

In one embodiment of the method according to the invention, the method uses a single analysis by mass spectrometry using a MALDI ionization technique by a single ionization of an analysis zone, and using a single calibration for the analysis.

The description given hereunder, referring to the appended figures, makes it possible to understand the invention better, without limiting the subject matter of the invention.

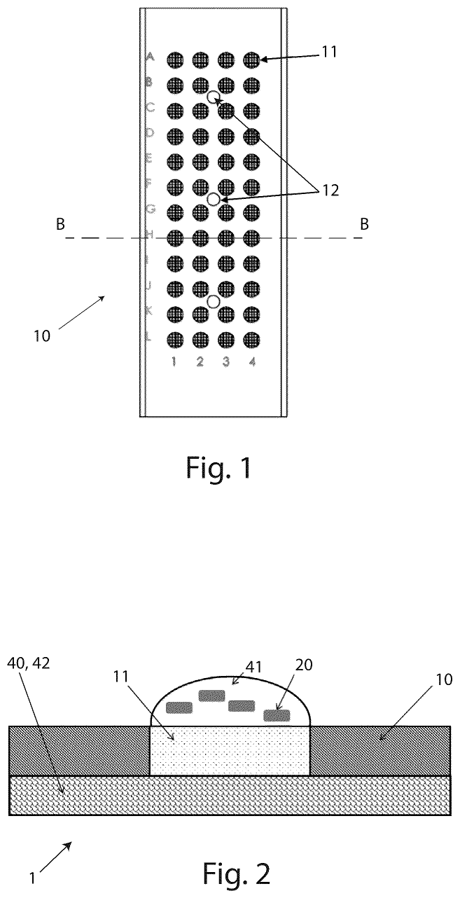

FIG. 1 is a schematic top view of an analysis plate according to the invention.

FIG. 2 is a cross section along the axis B-B of the analysis plate according to the invention during the incubation step.

FIG. 3 is a view in cross section along the axis B-B of the analysis plate according to the invention during the step of removing excess liquid according to a first embodiment,

FIG. 4 is a view in cross section along the axis B-B of the analysis plate according to the invention during the step of removing excess liquid according to a second embodiment,

FIG. 5 is a view in cross section along the axis B-B of the analysis plate according to the invention after the step of removing excess liquid regardless of the embodiment,

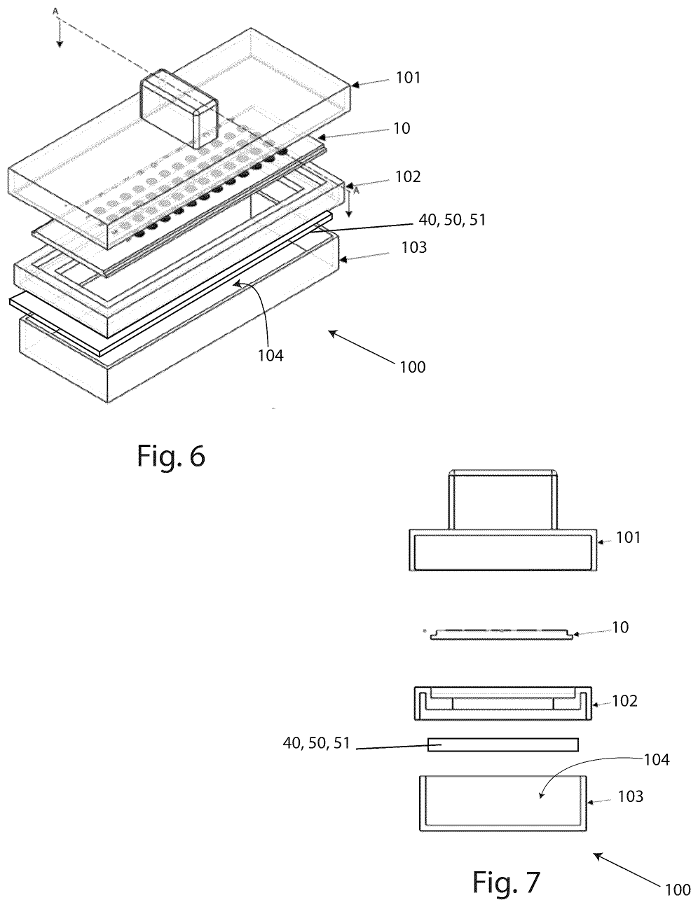

FIG. 6 is an exploded perspective view of a system according to the invention comprising a cassette containing an analysis plate according to the invention,

FIG. 7 is a cross section along the axis A-A of the system according to the invention shown in FIG. 6.

Firstly, the analysis plate according to the invention will be described referring to FIG. 1. FIG. 1 shows a model of analysis plate 10 according to the invention suitable for analysis by mass spectrometry. Said analysis plate 10 comprises 48 analysis zones 11 and three reference analysis zones 12. The analysis zones 11 are spaced and are arranged so that they are separate from one another. The reference analysis zones 12 are positioned between the arrays of analysis zones 11. More particularly in the example illustrated in FIG. 1, the analysis plate 10 comprises four columns (1-4) of twelve arrays (A-L) of analysis zones 11. Each analysis zone 11 is of circular shape and is configured to circumscribe one or more drops of populations of microorganism previously sampled. According to the invention, the analysis plate 10 according to the invention is made at least of two different materials: one for the analysis zones 11, 12 and one suitable for MALDI ionization and forming the analysis plate 10.

Secondly, referring to FIGS. 2 to 5, we shall describe certain steps of the method of characterization according to the invention by describing the analysis plate according to the invention.

More particularly, in FIG. 2, the analysis plate 10 according to the invention is arranged on an incubation support 40 humidified either by an incubation element 42 or by the culture medium 41 allowing growth of the microorganisms and disposed on each analysis zone 11, 12. As can be seen, the culture medium 41 is deposited on an analysis zone 11 on which a microbial agent (not shown) and a population of microorganisms 20 have already been deposited. The culture medium 41 as well as the preceding deposits are circumscribed by the analysis zone 11 owing to the porous material of said analysis zone 11. This configuration illustrated in FIG. 2 therefore illustrates the system 1 according to the invention during the incubation step.

In FIGS. 3 and 4, the incubation support 40 or the incubation element 42 is removed and is replaced, under the analysis plate 10, with an aspirating device 50, 51 for removing excess liquid. The aspirating device 50, 51 may be in the form of a vacuum aspirating device 50 (FIG. 3) or else in the form of blotting paper 51 (FIG. 4). FIG. 5 shows the analysis plate 10 according to the invention once the excess liquid has been removed: the microorganisms 20 are retained naturally on the surface of the analysis zone 11, without being able to pass through it owing to the size of the surface pores. The underside of the analysis plate 10 thus remains free from any potentially infectious microorganism.

Referring to FIGS. 6 and 7, a cassette 100 is described, in which the analysis plate 10 according to the invention is integrated. The cassette 100 comprises a cover 101, a gasket 102 on which the analysis plate 10 will be positioned and a container 103 having an open internal receiving space 104 on the underside of the analysis plate 10. Said internal space 104 of the container 103 is configured for receiving, completely or partially, an incubation support 40 or an incubation element 42 or else an aspirating device 50, 51.

The protocol for use of this cassette may be described by the following steps:
remove the cover from the cassette,
position a humid porous material inside the container,
place the gasket above the container,
position an analysis plate on the gasket,
deposit 2 μl of an inoculum comprising at least one microorganism population on each analysis zone bearing the antibiotic,
replace the cover, clipping it on the gasket,
incubate the cassette at 37° C. for 2 h (or more),
remove the assembly formed by the cover, the gasket and the analysis plate and put it on a second container containing a dry absorbent porous material, such as absorbent blotting paper,
leave the blotting paper to aspirate, by capillarity, all of the drops of liquids present on the analysis plate,
remove the cover,
add 1 μl of an HCCA matrix on the analysis zones bearing both the antibiotic and the bacteria,
remove the analysis plate and finalize its drying in the open air,
insert the analysis plate in a MALDI ionization source for analysis by mass spectrometry.

The following examples illustrate the invention but do not have any limiting character. The analyses were carried out with the VITEK®MS equipment marketed by the company bioMérieux. The analyses were carried out, in each of the following examples, at the end of acquisition. The spectra were acquired on all the characterization zones and were then analyzed:
for identification, the spectrum was analyzed using the VITEK®MS calculation engine and the database V3.2.0.

### Example 1: Isolation of Bacterial Microorganisms and Identification Using VITEK-MS

The experiment was carried out using the following steps:
12 microorganisms were isolated on Columbia blood agar (reference 43041, bioMérieux) after culture overnight at 37° C.
These microorganisms were deposited on the analysis zones of a VITEK®MS analysis plate (reference 410893, bioMérieux).
1 μl of HCCA matrix, alpha-cyano 4-hydroxycinnamic acid (VITEK®MS-CHCA, bioMérieux reference 411071) was deposited on the analysis zones already comprising the microorganisms.
The analysis plates were left to dry in accordance with the supplier's recommendations.
An analysis by MALDI-TOF mass spectrometer was carried out with VITEK®MS equipment (reference 4700563, bioMérieux).

The results were revealed in Myla (bioMérieux) and are reported in Table 1.

TABLE 1
Microorganisms identified

| | Microorganisms | Identification |
|---|---|---|
| Isolate | identified | probability (%) |
| Isolate 1 | Escherichia coli | 99.9 |
| Isolate 2 | Escherichia coli | 99.9 |
| Isolate 3 | Escherichia coli | 99.9 |
| Isolate 4 | Escherichia coli | 99.9 |
| Isolate 5 | Escherichia coli | 99.9 |
| Isolate 6 | Klebsiella pneumoniae | 99.9 |
| Isolate 7 | Klebsiella pneumoniae | 99.9 |
| Isolate 8 | Klebsiella pneumoniae | 99.9 |
| Isolate 9 | Staphylococcus aureus | 99.9 |
| Isolate 10 | Staphylococcus aureus | 99.9 |
| Isolate 11 | Staphylococcus aureus | 99.9 |
| Isolate 12 | Staphylococcus epidermidis | 99.9 |

### Example 2: Preparation of Analysis Plates According to the Invention with Antibiotics at Various Concentrations

Various solutions of antibiotic were deposited on analysis plates having the same geometry as the VITEK®MS targets (reference 410893, bioMérieux), but having analysis zones made of a porous material. More precisely, 48 analysis zones, made of porous material, distributed in 3 groups of 16 zones. These zones are arranged in 4 columns of 12 rows. Each column is identified by a numeral from 1 to 4. Each row is identified by a letter from A to L. Thus, analysis zone 13 is located at the intersection of the 9th row and 3rd column. The analysis plate also comprises 3 calibration positions, respectively at the center of the 3 groups of 16 analysis zones. In contrast to the zones intended for the samples, these calibration zones are not made of porous material.

The following solutions of antibiotic were prepared in water:
solution of oxacillin (Sigma reference 28221) at 0.125 μg/ml, 0.25 μg/ml, 0.5 μg/ml, 1 μg/ml, 2 μg/ml, 4 μg/ml, 8 μg/ml, 16 μg/ml, 32 μg/ml, 64 μg/ml, 128 μg/ml and 256 μg/ml;
solution of amoxicillin (Sigma reference A8523) at 2 μg/ml;
solution of ceftriaxone (Sigma reference C5793) at 24 μg/ml;
solution of meropenem (Sigma reference M2574) at 2 μg/ml.

2 μl of each solution of antibiotic was deposited in the form of a drop, on the analysis zones of analysis plates. The analysis plates were incubated at 37° C. in dry atmosphere, until the drops were completely dry. These analysis plates, thus functionalized, were stored at 4° C. in a plastic blister away from moisture and light until they were used.

Some analysis plates were functionalized entirely with only oxacillin at 2 μg/ml.

Other analysis plates were functionalized with 4 deposits of oxacillin at 0.125 μg/ml, 4 deposits of oxacillin at 0.25 μg/ml, 4 deposits of oxacillin at 0.5 μg/ml, 4 deposits of oxacillin at 1 μg/ml, 4 deposits of oxacillin at 2 μg/ml, 4 deposits of oxacillin at 4 μg/ml, 4 deposits of oxacillin at 8 μg/ml, 4 deposits of oxacillin at 16 μg/ml, 4 deposits of oxacillin at 32 μg/ml, 4 deposits of oxacillin at 64 μg/ml, 4 deposits of oxacillin at 128 μg/ml, 4 deposits of oxacillin at 256 μg/ml.

Finally, some analysis plates were functionalized with 16 deposits of amoxicillin at 2 μg/ml, 16 deposits of ceftriaxone at 24 μg/ml, 16 deposits of meropenem at 2 μg/ml.

This preliminary functionalization of the analysis plates is thus particularly advantageous since it makes it possible to provide a target that is ready to use, preferably manufactured industrially.

It is also possible to manufacture analysis plates in advance comprising 48 activated zones with the same antibiotic in order to test several microorganisms simultaneously. It is even possible to manufacture analysis plates functionalized with combinations and concentrations of antibiotic adapted to the nature of the microorganisms and/or to the regulatory and epidemic context of a region, or even of a given laboratory, as is the conventional practice for the applicant's VITEK®2 maps. An analysis plate with a combination of various antibiotics thus allows simultaneous characterization of the properties of resistance/sensitivity of a microorganism to a plurality of antibiotics. Similarly, the presence of analysis zones with different concentrations of antibiotic makes it possible to predict the minimum inhibitory concentration (MIC) of a microorganism.

### Example 3: Optimization of the Characterization of Antibiotic Resistance According to the Invention

Analysis plates, functionalized according to example 2, were placed in a cassette, as illustrated in FIGS. 3 to 5, to allow handling thereof. This cassette comprises a gasket so as to be able to dispose the analysis plate on a sponge impregnated to saturation with Muller-Hinton culture medium. The sponge impregnated with a culture medium serves as a support, and it is made of porous material as described in the description.

The minimum inhibitory concentration (MIC in μg/ml) was determined by microdilution in broth, a technique very widely used by a person skilled in the art.

The reference strains shown in Table 2 were analyzed.

TABLE 2
characteristics of the reference strains used.

| | | Minimum inhibitory concentration | | | | | |
|---|---|---|---|---|---|---|---|
| Reference | | (MIC in μg/ml) | | | | Phenotype | Mechanism of |
| strain | | Oxacillin | Amoxicillin | Ceftriaxone | Meropenem | (R = resistant, | resistance |
| No. | Species | (O) | (A) | (C) | (M) | S = sensitive) | identified by PCR |
| Ref 1 | K. | — | >128 | 64 | 64 | RA, RC, | KPC-2 |
| | pneumoniae | | | | | RM | |
| Ref 2 | K. | — | 64 | 32 | 0.25 | RA, RC, | OXA-48 |
| | pneumoniae | | | | | SM | |
| Ref 3 | E. coli | — | 32 | 16 | 0.5 | RA, RC, | TEM-2 |
| | | | | | | SM | |
| Ref 4 | E. coli | — | 1 | 0.25 | 0.06 | SA, SC, | — |
| | | | | | | SM | |
| Ref 5 | E. coli | — | >128 | 8 | 0.5 | RA, RC, | CTX-M |
| | | | | | | SM | |
| Ref 6 | S. | 0.25 | — | — | — | SO | — |
| | aureus | | | | | | |
| Ref 7 | S. | 128 | — | — | — | RO | mecA |
| | aureus | | | | | | |

In the Phenotype column, RA signifies resistance to amoxicillin, RC signifies resistance to ceftriaxone, RM signifies resistance to meropenem, RO resistance to oxacillin, SA signifies sensitive to amoxicillin, SC signifies sensitive to ceftriaxone, SM signifies sensitive to meropenem, SO sensitive to oxacillin.

The reference strains No. 1 to 7 were diluted to 0.5 McFarland (McF) in Muller-Hinton medium (bioMérieux reference AEB 110699), and then diluted again to 1/10°, 1/50° and 1/100°, always in Muller-Hinton medium.

For each dilution of microorganisms, 2 μl drops were deposited on 4 analysis zones of an analysis plate according to the invention. The dilutions of E. coli and of K. pneumoniae were analyzed on analysis plates with 16 deposits of amoxicillin, 16 deposits of ceftriaxone and 16 deposits of meropenem. The dilutions of S. aureus were analyzed on analysis plates with 2 μg/ml of oxacillin.

Control analysis plates, i.e. not functionalized (without antibiotic), were also prepared. The negative controls were treated as explained below, eliminating the step of incubation in the stove. In other words, the drops were aspirated immediately after deposition. The positive controls were treated at every point as explained below.

A cover was placed on the cassette containing the analysis plate so as to be clipped on the cassette and close the device without touching the top of the drops.

The analysis cassettes thus prepared were incubated in the stove at 37° C. for 2, 3, 4 or 5 hours.

At the end of the incubation step, the analysis plates are placed on very absorbent, dry blotting paper, with an absorption capacity of 25 μL·cm⁻², so that the drops present on the analysis plate are aspirated by capillarity through the analysis zones made of porous material.

The cover is then unclipped.

A calibrant (E. coli strain ATCC 8739 cultured on Columbia blood agar, reference 43041, bioMérieux) was deposited on the calibration zones.

1 μl of HCCA matrix, alpha-cyano 4-hydroxycinnamic acid (VITEK®MS-CHCA, bioMérieux reference 411071) was deposited on the analysis zones, comprising the microorganisms and the antimicrobial agent, and on the calibration zones comprising the calibrant.

The analysis plate is then dried and analyzed by VITEK®MS as described in example 1.

The dilution of the microorganisms was selected to give an identification probability less than or equal to 60% on the negative control plates, or even, most often, absence of identification (whatever the reason indicated by VITEK®MS). No identification with a species other than those studied was observed, otherwise it would have been regarded as an invalid result. Dilutions to 1/100° and to 1/10° were retained for the Gram-negative strains and for the Gram-positive strains, respectively.

The incubation time was selected to give an identification probability greater than 80% on the positive control targets. In general, said probability was observed after 2 hours of incubation for the Gram-negative strains and after 4 h for the Gram-positive strains. However, compliant results were obtained for all the strains after 4 h of incubation and it seemed simpler to use the same incubation time for all the microorganisms.

In these conditions, namely 4 h of incubation for a dilution to 1/100° and to 1/10° of the Gram-negative and Gram-positive strains respectively, identifications with a probability greater than or equal to 90% were observed for at least 3 deposits out of 4 when the reference microorganism was resistant to the antibiotic. Conversely, absences of identification or identifications with a probability lower than 90% were observed for at least 3 deposits out of 4 when the reference microorganism was sensitive to the antibiotic tested. These results indicate that, advantageously, an identification probability greater than or equal to 90% for at least 3 deposits out of 4 may be used as the threshold for differentiating the resistant strains from the sensitive strains.

It is possible, however, that certain species have a different behavior and require optimization of the analysis conditions (dilutions, incubation time) or of interpretation (identification probability) different than those observed for the species studied here.

### Example 4: Characterization of Resistance to Antibiotics According to the Invention

The isolates identified in example 1 were diluted to 0.5 McFarland (McF) in Muller-Hinton medium (bioMérieux reference AEB 110699), then diluted again to 1/100° for the Gram-negative bacteria and to 1/10° for the Gram-positive bacteria, always in Muller-Hinton medium.

Drops of 2 μl of the final dilutions were deposited on the analysis zones of the analysis plates according to the invention as described in example 2. As before, the dilutions of E. coli and of K. pneumoniae were analyzed on the targets with 16 deposits of amoxicillin, 16 deposits of ceftriaxone and 16 deposits of meropenem. The dilutions of Staphylococci were analyzed on the targets with 2 μg/ml of oxacillin.

When no sample deposit was provided on an analysis zone, this zone is covered with a Parafilm M plastic film (Bemis North America) before putting the analysis plate in the cassette. In this way, the analysis zone was not brought into contact with the culture medium contained in the sponge impregnated to saturation. The positions unused in the first analysis thus remained usable for a subsequent analysis.

The analysis was carried out according to the same procedure as in example 3 with an incubation of 4 h for all the microorganisms. Moreover, the results were interpreted as in example 3, i.e. with a threshold of identification probability greater than or equal to 90% for at least 3 deposits out of 4.

The results are reported in Tables 3 to 6 below. These results show that correct identification of the bacteria by MALDI-TOF could be obtained from the first series of acquisitions for all the analysis zones, with a level of confidence of 99.9%, showing that the experimental conditions again make it possible to differentiate the various species.

TABLE 3
Analysis of isolates 1 to 4 with an amoxicillin,
ceftriaxone and meropenem target

| | | | | | | Identi- |
|---|---|---|---|---|---|---|
| | | | | | | fication |
| | | Concen- | | | Microor- | proba- |
| Posi- | | tration | | Dilu- | ganisms | bility |
| tion | Antibiotic | (μg/ml) | Sample | tion | identified | (%) |
| A1 | Amoxicillin | 2 | Isolate 1 | 1/100° | E. coli | 99.9 |
| A2 | Amoxicillin | 2 | Isolate 1 | 1/100° | E. coli | 99.9 |
| A3 | Amoxicillin | 2 | Isolate 1 | 1/100° | E. coli | 99.9 |
| A4 | Amoxicillin | 2 | Isolate 1 | 1/100° | E. coli | 99.9 |
| B1 | Amoxicillin | 2 | Isolate 2 | 1/100° | — | — |
| B2 | Amoxicillin | 2 | Isolate 2 | 1/100° | E. coli | 60 |
| B3 | Amoxicillin | 2 | Isolate 2 | 1/100° | — | — |
| B4 | Amoxicillin | 2 | Isolate 2 | 1/100° | — | — |
| C1 | Amoxicillin | 2 | Isolate 3 | 1/100° | E. coli | 99.9 |
| C2 | Amoxicillin | 2 | Isolate 3 | 1/100° | E. coli | 99.9 |
| C3 | Amoxicillin | 2 | Isolate 3 | 1/100° | E. coli | 99.9 |
| C4 | Amoxicillin | 2 | Isolate 3 | 1/100° | E. coli | 99.9 |
| D1 | Amoxicillin | 2 | Isolate 4 | 1/100° | E. coli | 99.9 |
| D2 | Amoxicillin | 2 | Isolate 4 | 1/100° | E. coli | 99.9 |
| D3 | Amoxicillin | 2 | Isolate 4 | 1/100° | E. coli | 99.9 |
| D4 | Amoxicillin | 2 | Isolate 4 | 1/100° | E. coli | 99.9 |
| E1 | Ceftriaxone | 24 | Isolate 1 | 1/100° | — | — |
| E2 | Ceftriaxone | 24 | Isolate 1 | 1/100° | — | — |
| E3 | Ceftriaxone | 24 | Isolate 1 | 1/100° | E. coli | 99.9 |
| E4 | Ceftriaxone | 24 | Isolate 1 | 1/100° | — | — |
| F1 | Ceftriaxone | 24 | Isolate 2 | 1/100° | — | — |
| F2 | Ceftriaxone | 24 | Isolate 2 | 1/100° | — | — |
| F3 | Ceftriaxone | 24 | Isolate 2 | 1/100° | — | — |
| F4 | Ceftriaxone | 24 | Isolate 2 | 1/100° | — | — |
| G1 | Ceftriaxone | 24 | Isolate 3 | 1/100° | E. coli | 99.9 |
| G2 | Ceftriaxone | 24 | Isolate 3 | 1/100° | E. coli | 99.9 |
| G3 | Ceftriaxone | 24 | Isolate 3 | 1/100° | E. coli | 99.9 |
| G4 | Ceftriaxone | 24 | Isolate 3 | 1/100° | — | — |
| H1 | Ceftriaxone | 24 | Isolate 4 | 1/100° | E. coli | 99.9 |
| H₂ | Ceftriaxone | 24 | Isolate 4 | 1/100° | E. coli | 99.9 |
| H3 | Ceftriaxone | 24 | Isolate 4 | 1/100° | E. coli | 99.9 |
| H4 | Ceftriaxone | 24 | Isolate 4 | 1/100° | E. coli | 99.9 |
| I1 | Meropenem | 2 | Isolate 1 | 1/100° | — | — |
| I2 | Meropenem | 2 | Isolate 1 | 1/100° | — | — |
| I3 | Meropenem | 2 | Isolate 1 | 1/100° | — | — |
| I4 | Meropenem | 2 | Isolate 1 | 1/100° | — | — |
| J1 | Meropenem | 2 | Isolate 2 | 1/100° | — | — |
| J2 | Meropenem | 2 | Isolate 2 | 1/100° | — | — |
| J3 | Meropenem | 2 | Isolate 2 | 1/100° | — | — |
| J4 | Meropenem | 2 | Isolate 2 | 1/100° | — | — |
| K1 | Meropenem | 2 | Isolate 3 | 1/100° | — | — |
| K2 | Meropenem | 2 | Isolate 3 | 1/100° | — | — |
| K3 | Meropenem | 2 | Isolate 3 | 1/100° | — | — |
| K4 | Meropenem | 2 | Isolate 3 | 1/100° | — | — |
| L1 | Meropenem | 2 | Isolate 4 | 1/100° | E. coli | 99.9 |
| L2 | Meropenem | 2 | Isolate 4 | 1/100° | E. coli | 99.9 |
| L3 | Meropenem | 2 | Isolate 4 | 1/100° | E. coli | 99.9 |
| L4 | Meropenem | 2 | Isolate 4 | 1/100° | E. coli | 99.9 |

The results in Table 3 show that isolate 1 is characterized as resistant to amoxicillin, but sensitive to ceftriaxone and to meropenem. Isolate 2 is sensitive to the three antibiotics. Isolate 3 is resistant to amoxicillin and to ceftriaxone but sensitive to meropenem. Finally, isolate 4 is resistant to the three antibiotics.

These results demonstrate that it is possible to characterize the resistance of several microorganisms, with respect to several antibiotics, on one and the same analysis plate of the invention.

TABLE 4
Analysis of isolates 5 to 8 with an amoxicillin,
ceftriaxone and meropenem target

| | | | | | | Identi- |
|---|---|---|---|---|---|---|
| | | | | | | fication |
| | | Concen- | | | Microor- | proba- |
| Posi- | | tration | | Dilu- | ganisms | bility |
| tion | Antibiotic | (μg/ml) | Sample | tion | identified | (%) |
| A1 | Amoxicillin | 2 | Isolate 5 | 1/100° | E. coli | 99.9 |
| A2 | Amoxicillin | 2 | Isolate 5 | 1/100° | E. coli | 99.9 |
| A3 | Amoxicillin | 2 | Isolate 5 | 1/100° | E. coli | 99.9 |
| A4 | Amoxicillin | 2 | Isolate 5 | 1/100° | E. coli | 99.9 |
| B1 | Amoxicillin | 2 | Isolate 6 | 1/100° | K. pneu- | 99.9 |
| | | | | | moniae | |
| B2 | Amoxicillin | 2 | Isolate 6 | 1/100° | K. pneu- | 99.9 |
| | | | | | moniae | |
| B3 | Amoxicillin | 2 | Isolate 6 | 1/100° | K. pneu- | 99.9 |
| | | | | | moniae | |
| B4 | Amoxicillin | 2 | Isolate 6 | 1/100° | K. pneu- | 99.9 |
| | | | | | moniae | |
| C1 | Amoxicillin | 2 | Isolate 7 | 1/100° | K. pneu- | 99.9 |
| | | | | | moniae | |
| C2 | Amoxicillin | 2 | Isolate 7 | 1/100° | K. pneu- | 99.9 |
| | | | | | moniae | |
| C3 | Amoxicillin | 2 | Isolate 7 | 1/100° | K. pneu- | 99.9 |
| | | | | | moniae | |
| C4 | Amoxicillin | 2 | Isolate 7 | 1/100° | K. pneu- | 99.9 |
| | | | | | moniae | |
| D1 | Amoxicillin | 2 | Isolate 8 | 1/100° | K. pneu- | 99.9 |
| | | | | | moniae | |
| D2 | Amoxicillin | 2 | Isolate 8 | 1/100° | K. pneu- | 99.9 |
| | | | | | moniae | |
| D3 | Amoxicillin | 2 | Isolate 8 | 1/100° | K. pneu- | 99.9 |
| | | | | | moniae | |
| D4 | Amoxicillin | 2 | Isolate 8 | 1/100° | K. pneu- | 99.9 |
| | | | | | moniae | |
| E1 | Ceftriaxone | 24 | Isolate 5 | 1/100° | — | — |
| E2 | Ceftriaxone | 24 | Isolate 5 | 1/100° | — | — |
| E3 | Ceftriaxone | 24 | Isolate 5 | 1/100° | — | — |
| E4 | Ceftriaxone | 24 | Isolate 5 | 1/100° | — | — |
| F1 | Ceftriaxone | 24 | Isolate 6 | 1/100° | K. pneu- | 99.9 |
| | | | | | moniae | |
| F2 | Ceftriaxone | 24 | Isolate 6 | 1/100° | K. pneu- | 99.9 |
| | | | | | moniae | |
| F3 | Ceftriaxone | 24 | Isolate 6 | 1/100° | K. pneu- | 99.9 |
| | | | | | moniae | |
| F4 | Ceftriaxone | 24 | Isolate 6 | 1/100° | K. pneu- | 99.9 |
| | | | | | moniae | |
| G1 | Ceftriaxone | 24 | Isolate 7 | 1/100° | — | — |
| G2 | Ceftriaxone | 24 | Isolate 7 | 1/100° | — | — |
| G3 | Ceftriaxone | 24 | Isolate 7 | 1/100° | — | — |
| G4 | Ceftriaxone | 24 | Isolate 7 | 1/100° | — | — |
| H1 | Ceftriaxone | 24 | Isolate 8 | 1/100° | K. pneu- | 99.9 |
| | | | | | moniae | |
| H₂ | Ceftriaxone | 24 | Isolate 8 | 1/100° | K. pneu- | 99.9 |
| | | | | | moniae | |
| H3 | Ceftriaxone | 24 | Isolate 8 | 1/100° | K. pneu- | 99.9 |
| | | | | | moniae | |
| H4 | Ceftriaxone | 24 | Isolate 8 | 1/100° | K. pneu- | 99.9 |
| | | | | | moniae | |
| I1 | Meropenem | 2 | Isolate 5 | 1/100° | — | — |
| I2 | Meropenem | 2 | Isolate 5 | 1/100° | — | — |
| I3 | Meropenem | 2 | Isolate 5 | 1/100° | — | — |
| I4 | Meropenem | 2 | Isolate 5 | 1/100° | — | — |
| J1 | Meropenem | 2 | Isolate 6 | 1/100° | K. pneu- | 99.9 |
| | | | | | moniae | |
| J2 | Meropenem | 2 | Isolate 6 | 1/100° | K. pneu- | 99.9 |
| | | | | | moniae | |
| J3 | Meropenem | 2 | Isolate 6 | 1/100° | K. pneu- | 99.9 |
| | | | | | moniae | |
| J4 | Meropenem | 2 | Isolate 6 | 1/100° | K. pneu- | 99.9 |
| | | | | | moniae | |
| K1 | Meropenem | 2 | Isolate 7 | 1/100° | — | — |
| K2 | Meropenem | 2 | Isolate 7 | 1/100° | — | — |
| K3 | Meropenem | 2 | Isolate 7 | 1/100° | — | — |
| K4 | Meropenem | 2 | Isolate 7 | 1/100° | — | — |
| L1 | Meropenem | 2 | Isolate 8 | 1/100° | — | — |
| L2 | Meropenem | 2 | Isolate 8 | 1/100° | — | — |
| L3 | Meropenem | 2 | Isolate 8 | 1/100° | — | — |
| L4 | Meropenem | 2 | Isolate 8 | 1/100° | — | — |

Isolate 5 is thus characterized as an isolate of E. coli resistant to amoxicillin, but sensitive to ceftriaxone and to meropenem. Isolate 6 is an isolate of K. pneumoniae resistant to the 3 antibiotics. Isolate 7 is an isolate of K. pneumoniae resistant to amoxicillin, but sensitive to ceftriaxone and to meropenem. Isolate 8 is an isolate of K. pneumoniae resistant to amoxicillin and to ceftriaxone, but sensitive to meropenem.

Once again, the inventors have characterized the resistance of several microorganisms, including of different species, with respect to several antibiotics, on one and the same analysis plate.

It is also possible to characterize several microorganisms on a partially used target, as presented in Table 5 below.

TABLE 5
Analysis of isolates 9 to 12 with an oxacillin target

| | | | | | | Identi- |
|---|---|---|---|---|---|---|
| | | | | | | fication |
| | | Concen- | | | Microor- | proba- |
| Posi- | | tration | | Dilu- | ganisms | bility |
| tion | Antibiotic | (μg/ml) | Sample | tion | identified | (%) |
| A1 | Oxacillin | 2 | Isolate 9 | 1/10° | S. aureus | 99.9 |
| A2 | Oxacillin | 2 | Isolate 9 | 1/10° | S. aureus | 99.9 |
| A3 | Oxacillin | 2 | Isolate 9 | 1/10° | S. aureus | 99.9 |
| A4 | Oxacillin | 2 | Isolate 9 | 1/10° | S. aureus | 99.9 |
| B1 | Oxacillin | 2 | Isolate 10 | 1/10° | — | — |
| B2 | Oxacillin | 2 | Isolate 10 | 1/10° | — | — |
| B3 | Oxacillin | 2 | Isolate 10 | 1/10° | — | — |
| B4 | Oxacillin | 2 | Isolate 10 | 1/10° | — | — |
| C1 | Oxacillin | 2 | Isolate 11 | 1/10° | — | — |
| C2 | Oxacillin | 2 | Isolate 11 | 1/10° | — | — |
| C3 | Oxacillin | 2 | Isolate 11 | 1/10° | — | — |
| C4 | Oxacillin | 2 | Isolate 11 | 1/10° | — | — |
| D1 | Oxacillin | 2 | Isolate 12 | 1/10° | — | — |
| D2 | Oxacillin | 2 | Isolate 12 | 1/10° | — | — |
| D3 | Oxacillin | 2 | Isolate 12 | 1/10° | — | — |
| D4 | Oxacillin | 2 | Isolate 12 | 1/10° | — | — |
| E1 | Oxacillin | 2 | — | — | — | — |
| E2 | Oxacillin | 2 | — | — | — | — |
| E3 | Oxacillin | 2 | — | — | — | — |
| E4 | Oxacillin | 2 | — | — | — | — |
| F1 | Oxacillin | 2 | — | — | — | — |
| F2 | Oxacillin | 2 | — | — | — | — |
| F3 | Oxacillin | 2 | — | — | — | — |
| F4 | Oxacillin | 2 | — | — | — | — |
| G1 | Oxacillin | 2 | — | — | — | — |
| G2 | Oxacillin | 2 | — | — | — | — |
| G3 | Oxacillin | 2 | — | — | — | — |
| G4 | Oxacillin | 2 | — | — | — | — |
| H1 | Oxacillin | 2 | — | — | — | — |
| H₂ | Oxacillin | 2 | — | — | — | — |
| H3 | Oxacillin | 2 | — | — | — | — |
| H4 | Oxacillin | 2 | — | — | — | — |
| I1 | Oxacillin | 2 | — | — | — | — |
| I2 | Oxacillin | 2 | — | — | — | — |
| I3 | Oxacillin | 2 | — | — | — | — |
| I4 | Oxacillin | 2 | — | — | — | — |
| J1 | Oxacillin | 2 | — | — | — | — |
| J2 | Oxacillin | 2 | — | — | — | — |
| J3 | Oxacillin | 2 | — | — | — | — |
| J4 | Oxacillin | 2 | — | — | — | — |
| K1 | Oxacillin | 2 | — | — | — | — |
| K2 | Oxacillin | 2 | — | — | — | — |
| K3 | Oxacillin | 2 | — | — | — | — |
| K4 | Oxacillin | 2 | — | — | — | — |
| L1 | Oxacillin | 2 | — | — | — | — |
| L2 | Oxacillin | 2 | — | — | — | — |
| L3 | Oxacillin | 2 | — | — | — | — |
| L4 | Oxacillin | 2 | — | — | — | — |

Isolate 9 is thus characterized as an isolate of S. aureus resistant to oxacillin, whereas isolates 10 and 11 of S. aureus and isolate 12 of S. epidermidis are sensitive to it.

Note that positions E1 to L4 were not used in this analysis. They were protected by a Parafilm M plastic film and are usable for a new analysis (cf. Table 6).

TABLE 6
Second analysis of isolates 9 to 12 with
the same analysis plate as for Table 4

| | | | | | | Identi- |
|---|---|---|---|---|---|---|
| | | | | | | fication |
| | | Concen- | | | Microor- | proba- |
| Posi- | | tration | | Dilu- | ganisms | bility |
| tion | Antibiotic | (μg/ml) | Sample | tion | identified | (%) |
| A1 | Oxacillin | 2 | — | — | — | — |
| A2 | Oxacillin | 2 | — | — | — | — |
| A3 | Oxacillin | 2 | — | — | — | — |
| A4 | Oxacillin | 2 | — | — | — | — |
| B1 | Oxacillin | 2 | — | — | — | — |
| B2 | Oxacillin | 2 | — | — | — | — |
| B3 | Oxacillin | 2 | — | — | — | — |
| B4 | Oxacillin | 2 | — | — | — | — |
| C1 | Oxacillin | 2 | — | — | — | — |
| C2 | Oxacillin | 2 | — | — | — | — |
| C3 | Oxacillin | 2 | — | — | — | — |
| C4 | Oxacillin | 2 | — | — | — | — |
| D1 | Oxacillin | 2 | — | — | — | — |
| D2 | Oxacillin | 2 | — | — | — | — |
| D3 | Oxacillin | 2 | — | — | — | — |
| D4 | Oxacillin | 2 | — | — | — | — |
| E1 | Oxacillin | 2 | Isolate 9 | 1/10° | S. aureus | 99.9 |
| E2 | Oxacillin | 2 | Isolate 9 | 1/10° | S. aureus | 99.9 |
| E3 | Oxacillin | 2 | Isolate 9 | 1/10° | S. aureus | 99.9 |
| E4 | Oxacillin | 2 | Isolate 9 | 1/10° | S. aureus | 99.9 |
| F1 | Oxacillin | 2 | Isolate 10 | 1/10° | — | — |
| F2 | Oxacillin | 2 | Isolate 10 | 1/10° | — | — |
| F3 | Oxacillin | 2 | Isolate 10 | 1/10° | — | — |
| F4 | Oxacillin | 2 | Isolate 10 | 1/10° | — | — |
| G1 | Oxacillin | 2 | Isolate 11 | 1/10° | — | — |
| G2 | Oxacillin | 2 | Isolate 11 | 1/10° | — | — |
| G3 | Oxacillin | 2 | Isolate 11 | 1/10° | — | — |
| G4 | Oxacillin | 2 | Isolate 11 | 1/10° | — | — |
| H1 | Oxacillin | 2 | Isolate 12 | 1/10° | — | — |
| H₂ | Oxacillin | 2 | Isolate 12 | 1/10° | — | — |
| H3 | Oxacillin | 2 | Isolate 12 | 1/10° | — | — |
| H4 | Oxacillin | 2 | Isolate 12 | 1/10° | — | — |
| I1 | Oxacillin | 2 | — | — | — | — |
| I2 | Oxacillin | 2 | — | — | — | — |
| I3 | Oxacillin | 2 | — | — | — | — |
| I4 | Oxacillin | 2 | — | — | — | — |
| J1 | Oxacillin | 2 | — | — | — | — |
| J2 | Oxacillin | 2 | — | — | — | — |
| J3 | Oxacillin | 2 | — | — | — | — |
| J4 | Oxacillin | 2 | — | — | — | — |
| K1 | Oxacillin | 2 | — | — | — | — |
| K2 | Oxacillin | 2 | — | — | — | — |
| K3 | Oxacillin | 2 | — | — | — | — |
| K4 | Oxacillin | 2 | — | — | — | — |
| L1 | Oxacillin | 2 | — | — | — | — |
| L2 | Oxacillin | 2 | — | — | — | — |
| L3 | Oxacillin | 2 | — | — | — | — |
| L4 | Oxacillin | 2 | — | — | — | — |

The same results were obtained in Tables 5 and 6 in two successive analyses with the same isolates on the same analysis plate, which proves that an analysis zone partially used a first time can be reused a second time, which is advantageous for avoiding waste of analysis plates. This experiment also demonstrates the reproducibility of the technique.

### Example 5: Determination of the Minimum Inhibitory Concentration (MIC) According to the Invention

The dilutions of Staphylococci of the reference strains and of the isolates were analyzed on the analysis plates according to the invention with 4 deposits of oxacillin at 0.125 μg/ml, 4 deposits of oxacillin at 0.25 μg/ml, 4 deposits of oxacillin at 0.5 μg/ml, 4 deposits of oxacillin at 1 μg/ml, 4 deposits of oxacillin at 2 μg/ml, 4 deposits of oxacillin at 4 μg/ml, 4 deposits of oxacillin at 8 μg/ml, 4 deposits of oxacillin at 16 μg/ml, 4 deposits of oxacillin at 32 μg/ml, 4 deposits of oxacillin at 64 μg/ml, 4 deposits of oxacillin at 128 μg/ml, 4 deposits of oxacillin at 256 μg/ml.

Reference strain No. 6 was identified with a probability greater than 99.9% as S. aureus in 3 deposits out of 4 with a concentration of oxacillin of 0.125 μg/ml, for 1 deposit out of 4 for a concentration of 0.25 μg/ml but was not identified for the higher concentrations of oxacillin. This strain has an MIC of 0.25 μg/ml (cf. Table 2), which corresponds to the lowest growth inhibiting concentration observed by the method of the present invention.

Surprisingly, a similar phenomenon was observed with reference strain No. 7. S. aureus was identified with a probability of 99.9% of 0.125 at 64 μg/ml of oxacillin but was not identified for concentrations of 128 and 256 μg/ml. An MIC of 128, compliant with that in Table 2, is thus demonstrated.

Isolates 9 to 12 were analyzed in the same way and an MIC of 16 μg/ml was observed for isolate 9, and of 0.25, 0.5 and 0.125 respectively for isolates 10 to 12. These observations comply with the predictions of resistance to oxacillin in Example 4, but isolate 9 is resistant, in contrast to isolates 10 to 12. In addition, they allow finer characterization of the properties of resistance or of sensitivity of the microorganisms by determining the minimum inhibitory concentration (MIC).

Of course, the invention is not limited to the embodiments described and shown in the appended figures. Modifications are still possible, in particular from the standpoint of the constitution of the various elements or by substitution of technical equivalents, while remaining within the scope of protection of the invention.

1. An analysis plate configured to allow characterization of microorganisms by mass spectrometry, the analysis plate comprising at least one analysis zone configured for a biological sample containing a population of at least one microorganism, wherein part or the whole of the analysis zone is made of a porous material, the analysis zone comprising at least one antimicrobial agent.

2. The analysis plate as claimed in claim 1, wherein the pore size of the analysis zone is less than the size of the at least one microorganism to be characterized.

3. The analysis plate as claimed in claim 1, wherein the plate is made at least partially of polymer covered with a layer of stainless steel.

4. The analysis plate as claimed in claim 1, wherein the plate comprises a plurality of analysis zones, each analysis zone bearing at least one antimicrobial agent.

5. The analysis plate as claimed in claim 4, wherein at least one first analysis zone from the plurality bears a first antimicrobial agent, and a second analysis zone from the plurality, separate from the first analysis zone, bears a second antimicrobial agent different than the first antimicrobial agent.

6. A system comprising:
an analysis plate as claimed in claim 1, on which a population of at least one microorganism and a culture medium are deposited on at least one analysis zone and
an incubation element configured for maintaining the humidity of the at least one analysis zone,
the incubation element being positioned under the analysis plate.

7. A method for characterizing a population of at least one microorganism, the characterization comprising at least determination of the possible resistance of a population of a microorganism to at least one antimicrobial agent,
wherein the method comprises the following successive steps:
a step consisting of supplying an analysis plate as claimed in claim 1, for characterization of the population of at least one microorganism,
a step of depositing the population of the at least one microorganism in liquid form on the at least one analysis zone in contact with the antimicrobial agent previously deposited on the analysis zone,
an incubation step, consisting of storing the analysis plate in conditions and for a sufficient period to allow interaction of the at least one antimicrobial agent and of the at least one microorganism present,
a step of removing the liquid containing the population of the at least one microorganism by aspiration through the pores of the analysis zone,
a step of depositing, on the at least one analysis zone, a matrix suitable for the MALDI ionization technique,
a step of analysis, by mass spectrometry using a MALDI ionization technique, of a population of the at least one microorganism deposited on the analysis zone, making it possible to conclude whether a population of a microorganism resistant to the antimicrobial agent is present in the analysis zone.

8. The method of characterization as claimed in claim 7, wherein the system comprises an incubation support, made of porous material.

9. The method of characterization as claimed in claim 8, wherein the incubation support is moistened by a culture medium or by the incubation element.

10. The method of characterization as claimed in claim 7, wherein the incubation step is carried out in an incubation chamber.

11. The method of characterization as claimed in claim 7, wherein the incubation step is carried out for at least 2 hours.

12. The method of characterization as claimed in claim 7, wherein it comprises a step of determining the resistance of the microorganism to the antimicrobial agent by observing the presence of proteins of the microorganism in quantity such that it can be concluded that there is growth of the microorganism despite the presence of the antimicrobial agent during the incubation step.

13. The method of characterization as claimed in claim 7, wherein a population of a single microorganism to be characterized is deposited.

14. The method of characterization as claimed in claim 7, wherein the population of microorganism(s) is obtained after a step of concentration, enrichment and/or purification and/or corresponds to a colony or to a fraction of a colony obtained after growth on a suitable medium.

15. The method of characterization as claimed in claim 7, wherein the characterization comprises, in addition, identification of the genus, or of the species of a population of a microorganism deposited on the analysis zone.
